(19) United States
(12) Patent Application Publication
(10) Pub. No.: US 2009/0182139 A1
(43) Pub. Date: Jul. 16, 2009

(54) PROCESS FOR THE PREPARATION OF AN N-ALKYL LACTAM WITH IMPROVED COLOUR QUALITY

(75) Inventors: Tobias Wabnitz, Mannheim (DE); Rolf Pinkos, Bad Dürkheim (DE); Karl Ott, Plankstadt (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF SE, LUDWIGSHAFEN (DE)

(21) Appl. No.: 12/296,115

(22) PCT Filed: Mar. 29, 2007
(86) PCT No.: PCT/EP07/53008

§ 371 Date: Oct. 6, 2008

(30) Foreign Application Priority Data
Apr. 6, 2006 (EP) 06112323.8

(51) Int. Cl. C07D 201/08; C07D 207/267; C07D 201/02; C07D 211/06

(52) U.S. Cl. 540/533; 548/552; 546/243; 548/554

(57) ABSTRACT

A process for preparing an N-alkyllactam with improved color quality, wherein from 0.01 to 10% by weight of a C₁₋₁₀-alcohol or a compound which releases from 0.01 to 10% by weight of a C₁₋₁₀-alcohol is added to the N-alkyllactam.

A mixture comprising at least 99.0% by weight of an N-alkyllactam and in the range from 100 to 5000 ppm by weight of a C₁₋₁₀-alcohol or of an acetal, aminal or of an orthoester which releases in the range from 100 to 5000 ppm by weight of a C₁₋₁₀-alcohol.

The present invention relates to a process for preparing an N-alkyllactam with improved color quality and to mixtures comprising at least 99.0% by weight of an N-alkyllactam and in the range from 100 to 5000 ppm by weight of a C₁₋₁₀-alcohol, or of an acetal, aminal or of an orthoester which releases in the range from 100 to 5000 ppm by weight of a C₁₋₁₀-alcohol.

N-alkyllactams are important products in the chemicals industry. Most widespread among them are N-alkylpyrrolidones (N-alkyllactams with a five-membered ring).

N-alkylpyrrolidones are, for example, organic solvents which are used in a multitude of applications.

N-alkylpyrrolidones are thermally stable, chemically largely inert, colorless, low-viscosity and aprotic solvents with broad usability. For instance, N-methylpyrrolidone (NMP) and N-ethylpyrrolidone (NEP) and also the higher homologs are usable as solvents, diluents, extractants, detergents, degreasing agents, adsorbents and/or dispersants.

NMP finds use in the extraction of pure hydrocarbons in petrochemical processing, in the purification and removal of gases such as acetylene 1,3-butadiene or isoprene, in aromatics extraction, for example in the Distapex process of LURGI GmbH, in acid gas scrubbing and in lubricant oil extraction. Moreover, NMP can be used as a solvent for polymer dispersions, for example for polyurethane dispersions.

NMP is also a good solvent for many plastics such as polyvinyl chloride (PVC), polyurethanes (PU), acrylates or butadiene-acrylonitrile copolymers and is used in their processing.

NMP is also used as a detergent in the removal of paint and varnish residues, and also as a pickling agent and as a detergent for metal, ceramic, glass and plastic surfaces.

NMP is likewise a solvent or cosolvent for the formulation of active ingredients in crop protection.

NEP and other N-alkylpyrrolidones can replace NMP in many applications and additionally exhibit advantageous properties in many cases (WO-A-2005/090447, BASF AG).

Other widely used N-alkyllactams are N-alkylpiperidones and N-alkylcaprolactams. N-alkylcaprolactams, especially N-methylcaprolactam, can be used as selective solvents for gas deacidification, as described in Chem. Techn. 29 (1977), pages 445-448 (Wehner et al., VEB Leuna). N-alkylcaprolactams also find use in the extraction of hydrocarbons owing to the high achievable selectivities, as described in Chem. Techn. 27 (1975), pages 401-405 (Wehner et al., VEB Leuna). See also WO-A-05/092953 (BASF AG). N-alkylpiperidones, for example N-methylpiperidone, can likewise be used in these applications.

The preparation of N-alkyllactams is known. N-alkylpyrrolidones can be effected, for example, by reacting gamma-butyrolactone (γ-BL) with monoalkylamines to release one equivalent of water, for example analogously to Ullmann's Encyclopedia of Industrial Chemistry, volume A22, 5th ed., p. 459 (1993) or analogously to DE-A-19 626 123 (BASF AG). N-alkylpyrrolidones can likewise be prepared from maleic anhydride or other dicarboxylic acid derivatives and monoethylamines in the presence of hydrogen and a hydrogenation catalyst, for example according to EP-A-745 598 (Bayer AG) or WO-A-02/102773 (BASF AG).

Other N-alkyllactams such as N-alkylpiperidones and N-alkylccaprolactams can likewise be prepared from the corresponding lactones by reacting with monoalkylamines, as described, for example, by Yakugaku Zasshi 71 (1951), 1341 (Susagawa et al.). In addition, these lactams can also be obtained by reacting oxynitriles with monomethylamines, as disclosed in DE-A-11 92 208 (BASF AG), or else elegantly by reacting lactams with monoalcohols or dialkyl ethers over acidic catalysts such as Al₂O₃, as described in Chem. Techn. 33 (1981), 193-196 (Wehner et al., VEB Leuna) or RO 137218 (Centrul de Cercetari pentru Fibre Chimice), or else with other alkylating agents such as dialkyl sulfates or alkyl halides under basic conditions, as described, for example, in J. Org. Chem. 29 (1964), pages 2748-2750 (Moriarty).

Purification processes for N-alkyllactams are known. The purification of N-alkylpyrrolidones can be effected, for example, by fractional distillation (including multiple distillation, as described in JP 06 228 088 (Mitsubishi Kasei Corp.)) or by extraction. Other or additional purification steps may be treatment with ion exchangers as described, for example, in EP-A-1 038 867 (BASF AG), or with solid adsorbents such as aluminum oxide analogously to WO-A-2005/092851 (Lyondell L. P.). N-alkylpyrrolidones can also be purified in the presence of acids such as toluenesulfonic acid (described, for example, JP 11 071 346 (Tonen Corp.)) or phosphoric acid (described, for example, in JP 2028148 (Ouchi Shinko Chem.)) during distillation. Other advantageous additives during the preparation and/or distillation may be alkali metal, alkaline earth metal or ammonium borohydrides, as disclosed, for example, in U.S. Pat. No. 4,885,371 (GAF Chemicals Corp.), oxidizing agents such as potassium permanganate, sodium perborate or potassium dichromate as described in JP 72 22 225 (Teijin Ltd.), or sodium hydroxide as described in U.S. Pat. No. 2,964,535 (Monsanto Chemicals).

In addition, JP-A-2001 089 446 (Mitsubishi Chem. Corp.), teaches that clean NMP with low color can be obtained when the amounts of hydrogen and oxygen do not exceed limiting values of 0.01 mol % and 0.002 mol % based on the pyrrolidone content during distillation. According to JP 62 79 401 (Mitsubishi Kasei Corp.), colorless N-methylpyrrolidone can also be obtained by thermal treatment (heating at 150-250° C.) and subsequent distillation.

Other N-alkyllactams such as N-alkylpiperidones and N-alkylcaprolactams can be purified in analogous ways.

For many applications of N-alkyllactams, for example the use of N-alkylpyrrolidones as solvents in the preparation of paints and varnishes or adhesives or in the production of plastics, it is important that these solvents are used in substantially colorless, i.e. unyellowed form.

N-alkylpyrrolidones tend to yellow in the course of storage and are then no longer suitable for such applications, since some of the discoloration is preserved in the products such as varnishes or plastics and is undesired there.

Methods for removing yellowing by purification or other treatment of N-alkylpyrrolidones and for avoiding yellowing during storage are therefore important.

It is known that purification processes such as fractional distillations, multiple distillation, distillations in the presence of additives such as acids (e.g. p-toluenesulfonic acid), bases (e.g. sodium hydroxide), reducing agents (e.g. sodium borohydride) and oxidizing agents (e.g. potassium permanganate) can lead to an improvement, i.e. to a brightening, in the color of N-alkylpyrrolidones.

It is also known that the removal of impurities by treatment with aluminum oxide or macroporous ion exchange resins can lead to an improvement of product properties, including the color.

Moreover, it has already been stated that additives such as sodium borohydride, for example, can lead to improved color properties during the synthesis of N-alkylpyrrolidones.

While numerous methods of this type for improving the color properties have been described for purification and preparation processes, there are only very few technical solutions for stabilization of the product during storage.

JP-A-2003 081 885 (Mitsubishi Chem. Corp.) states that the saturation and blanketing of N-methylpyrrolidone with nitrogen, such that only very small amounts of oxygen remain, greatly improves the storage stability and significantly lowers the rate of oxidative degradation. The thermal stability toward decomposition at high temperatures (above 250° C.) of N-methylpyrrolidone can, according to U.S. Pat. No. 4,168,226 (Exxon Research & Engineering Co.) also be improved by adding up to 0.5% by weight of water. In these cases, the decomposition of NMP proceeds at a much slower rate.

It is an object of the present invention to discover a method for preventing and/or slowing the yellowing of N-alkyllactams during processing and storage. An improved, economically viable, easy-to-perform process shall be discovered for preparing an N-alkyllactam with improved color quality, i.e. reduction in the discoloration and/or improvement in the color stability, especially in the course of storage.

We have accordingly found a process for preparing an N-alkyllactam with improved color quality, which comprises adding in the range from 0.01 to 10% by weight of a C₁₋₁₀-alcohol or a compound which releases in the range from 0.01 to 10% by weight of a C₁₋₁₀-alcohol to the N-alkyllactam.

We have also found mixtures comprising at least 99.0% by weight of an N-alkyllactam and in the range from 100 to 5000 ppm by weight of a C₁₋₁₀-alcohol, or of an acetal, aminal or of an orthoester which releases in the range from 100 to 5000 ppm by weight of a C₁₋₁₀-alcohol.

The process preferably comprises adding to the N-alkyllactam in the range from 0.02 to 2% by weight, especially from 0.03 to 1% by weight, particularly from 0.03 to 0.5% by weight, more particularly from 0.03 to 0.2% by weight, more particularly from 0.03 to 0.1% by weight, of a C₁₋₁₀-alcohol, or a compound which releases in the range from 0.02 to 2% by weight, especially from 0.03 to 1% by weight, particularly from 0.03 to 0.5% by weight, more particularly from 0.03 to 0.2% by weight, more particularly from 0.03 to 0.1% by weight, of a C₁₋₁₀-alcohol.

The process according to the invention is preferably performed at a temperature in the range from −20 to 400° C., particularly in the range from 0 to 350° C., more particularly in the range from 10 to 250° C.

In further embodiments, the lower limit of a preferred temperature range is at 20, 50, 100 or 200° C., the upper limit at 220 or 150° C.

This influence of alcohols on the color development of N-alkyllactams that we have found is novel. In contrast to the technical teachings, for example of the thermal stabilization of N-methylpyrrolidone above 250° C. from U.S. Pat. No. 4,168,226 (Exxon Research & Engineering Co.), the addition of alcohol or the release of alcohol in accordance with the process according to the invention has no measurable positive influence on the rate of decomposition of the N-alkyllactams, but it does on their color quality, for example color evolution.

The precise mechanism of action of the alcohol additions is not yet known. According to the invention, all N-alkyllactams can be treated in this way. The lactam ring of the N-alkyllactams may, for example, have from four to eight carbon atoms, preferably four (pyrrolidones), five (piperidones) or six (caprolactams); more preferably, 2-pyrrolidones and 2-piperidones may be used.

In the process according to the invention, preference is given to using N-alkyllactams of the formula I

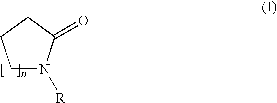

in which R is a
linear or branched, saturated aliphatic radical, preferably C₁₋₁₂-alkyl, such as methyl, ethyl, n-propyl, isopropyl, n-butyl, isobutyl, sec-butyl, tert-butyl, n-pentyl, isopentyl, sec-pentyl, neo-pentyl, 1,2-dimethylpropyl, n-hexyl, isohexyl, sec-hexyl, cyclopentylmethyl, n-heptyl, isoheptyl, cyclohexylmethyl, n-octyl, 2-ethylhexyl, n-nonyl, isononyl, n-decyl, isodecyl, n-undecyl, n-dodecyl, isododecyl, more preferably C₁₋₈-alkyl, such as methyl, ethyl, n-propyl, isopropyl, n-butyl, isobutyl, sec-butyl, tert-butyl, n-pentyl, n-hexyl, n-octyl and 2-ethylhexyl, most preferably C₁₋₄-alkyl, such as methyl, ethyl, n-propyl, isopropyl, n-butyl, isobutyl, sec-butyl, tert-butyl,
or a
saturated cycloaliphatic radical having from 3 to 12 carbon atoms, preferably C₄₋₈-cycloalkyl, such as cyclobutyl, cyclopentyl, cyclohexyl, cycloheptyl and cyclooctyl, more preferably cyclopentyl and cyclohexyl,
and n is an integer from 1 to 5
and where the carbon atoms of the heterocyclic ring of the N-substituted lactam may bear from one to two substituents inert under the conditions, for example alkyl radicals, e.g. C₁₋₈-alkyl radicals, which are each independently preferably a C₁₋₈-alkyl radical, particularly a C₁₋₄-alkyl radical.

Examples of C₁₋₈-alkyl radicals which may bear the carbon atoms of the heterocyclic ring of the N-substituted lactam are:

methyl, ethyl, n-propyl, isopropyl, n-butyl, isobutyl, sec-butyl, tert-butyl, n-pentyl, isopentyl and 2-ethylhexyl,
for example in 1,5-dimethyl-2-pyrrolidone and 1-ethyl-5-methyl-2-pyrrolidone.

In the process according to the invention, particular preference is given to using N-alkyllactams of the formula I

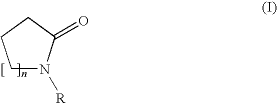

in which R is C₁₋₄-alkyl as described above and n is 1, 2 or 3, and where the carbon atoms of the heterocyclic ring of the N-substituted lactam may bear a C₁₋₄-alkyl radical, particularly methyl or ethyl radical.

The N-alkyllactams used may have a purity of ≧90% by weight, preferably ≧95% by weight, more preferably ≧99% by weight.

Possible impurities of the N-alkyllactams, which are not troublesome, are corresponding lactones (e.g. gamma-butyrolactone), corresponding N-unsubstituted lactams (e.g. pyrrolidone), organic peroxides, corresponding monoalkylamines (e.g. monoethylamine), corresponding cyclic N-alkylimides (e.g. N-alkylsuccinimides), water, which may preferably each be present in amounts of ≦1% by weight.

Useful alcohols which may be added or released to increase the color stability include all alcohols having one or more hydroxyl function(s), especially those having from 1 to 10, preferably from 1 to 8 carbon atoms, more preferably from 1 to 3 carbon atoms, most preferably from 1 to 2 carbon atoms.

Preferred monofunctional (monohydric) alcohols are methanol, ethanol, n-propanol, isopropanol, n-butanol, n-pentanol, n-hexanol, n-heptanol and n-octanol.

Particularly effective polyfunctional (polyhydric) alcohols are 1,2-ethylene glycol, 1,2-propanediol, 1,3-propanediol, glycerol, pentaerythritol and sorbitol.

Very particularly preferred added or released alcohols are methanol and 1,2-ethylene glycol.

In the context of the invention, it is also possible to add those substances which release alcohols only after a chemical conversion, for example with water (hydrolysis) or amines (aminolysis), which may be present in N-alkyllactams in traces or may be added.

These alcohol sources, also referred to hereinafter as alcohol precursors, include, for example, dimethoxymethane, diethoxymethane, tetramethoxymethane, tetraethoxymethane, trimethyl orthoformate and triethyl orthoformate, which, for example, release alcohols and esters or alcohols and formaldehyde by hydrolysis.

Of course, it is also possible to use a mixture of the alcohols and/or alcohol precursors. The purity of the added alcohols or alcohol precursors is not critical, and impurities such as water, ethers, esters and hydrocarbons, for example, are tolerated.

The alcohol can be added or alcohol precursor can be added in several ways; for example, the alcohols and/or alcohol precursors can be added to the N-alkyllactam directly after its synthesis, and also after or in the course of purification of the lactam. It is equally possible to add it when the lactam is transferred to storable or transportable containers. The addition of the alcohols or alcohol precursors can be effected either batchwise or continuously.

In accordance with the above remarks, the invention also provides

mixtures comprising at least 99.0% by weight, preferably ≧99.2% by weight, particularly ≧99.5% by weight, of an N-alkyllactam and
in the range from 100 to 5000 ppm by weight, preferably from 200 to 2000 ppm by weight, particularly from 300 to 1000 ppm by weight, of a C₁₋₁₀-alcohol, preferably C₁₋₈-alcohol, particularly C₁₋₃-alcohol, very particularly C₁₋₂-alcohol,
or of an acetal, aminal or of an orthoester which releases in the range from 100 to 5000 ppm by weight, preferably from 200 to 2000 ppm by weight, particularly from 300 to 1000 ppm by weight, of a C₁₋₁₀-alcohol, preferably C₁₋₈-alcohol, particularly C₁₋₃-alcohol, very particularly C₁₋₂-alcohol,
especially mixtures comprising at least 99.0% by weight, preferably ≧99.2% by weight, particularly ≧99.5% by weight, of N-ethyl-2-pyrrolidone (NEP) and in the range from 100 to 5000 ppm by weight, preferably from 200 to 2000 ppm by weight, particularly from 300 to 1000 ppm by weight, of methanol or 1,2-ethylene glycol,
especially mixtures comprising at least 99.0% by weight, preferably ≧99.2% by weight, particularly ≧99.5% by weight, of N-ethyl-ε-caprolactam (NEC) and in the range from 100 to 5000 ppm by weight, preferably from 200 to 2000 ppm by weight, particularly from 300 to 1000 ppm by weight, of methanol or 1,2-ethylene glycol,
especially mixtures comprising at least 99.0% by weight, preferably ≧99.2% by weight, particularly ≧99.5% by weight, of 1,5-dimethyl-2-pyrrolidone and in the range from 100 to 5000 ppm by weight, preferably from 200 to 2000 ppm by weight, particularly from 300 to 1000 ppm by weight, of methanol or 1,2-ethylene glycol,
especially mixtures comprising at least 99.0% by weight, preferably ≧99.2% by weight, particularly ≧99.5% by weight, of 1-ethyl-5-methyl-2-pyrrolidone and in the range from 100 to 5000 ppm by weight, preferably from 200 to 2000 ppm by weight, particularly from 300 to 1000 ppm by weight, of methanol or 1,2-ethylene glycol,
especially mixtures comprising at least 99.0% by weight, preferably ≧99.2% by weight, particularly ≧99.5% by weight, of 1-methyl-2-piperidone and in the range from 100 to 5000 ppm by weight, preferably from 200 to 2000 ppm by weight, particularly from 300 to 1000 ppm by weight, of methanol or 1,2-ethylene glycol.

All ppm data are based on the weight (ppm by weight).

### EXAMPLES

The APHA color numbers (Hazen) were determined to DIN EN ISO 6271.

GC method for determining the purity of the N-alkyllactams:

The lactams were injected undiluted into the GC chromatograph (from HP, carrier gas: hydrogen) onto a 30 m DB5 column (from J+W) and analyzed at oven temperatures of from 60° C. to 260° C. (heating rate 16 Kelvin per minute up to 220° C., then 20 Kelvin per minute up to 260° C.) with a flame ionization detector (temperature: 290° C.). The purity was determined by integrating the signals of the chromatogram.

The determination of the total content of alcohol in the N-alkyllactam, including the alcohols releasable by hydrolysis, was carried out by headspace gas chromatography. Before injection into the gas chromatograph, the samples (about 100 mg) were admixed with dilute aqueous phosphoric acid (about 1 ml) and heated to 80° C. for 1 h in order to release the entire amount of alcohol. The headspace was injected into a gas chromatograph (from HP, carrier gas: hydrogen) with a DB1 column (from J+W) and a flame ionization detector. The evaluation was effected by integrating the signals of the chromatogram. The calibration was effected by adding defined amounts of the alcohol to be analyzed and analysis by a similar method.

### Example 1

N-ethylpyrrolidone (250 ml, purity 99.69% by GC, Hazen color number: 12 APHA) was admixed with methanol (500 ppm) and heated to 100° C. in a 500 ml glass flask with attached reflux condenser and drying tube in the presence of air. After 72 hours (h), the purity had decreased (to 97.55% by GC), while the color number had risen to 208 APHA.

### Example 2

N-ethylpyrrolidone (250 ml, purity 99.69% by GC, Hazen color number: 12 APHA) was admixed with 1,2-ethylene glycol (500 ppm) and heated to 100° C. in a 500 ml glass flask with attached reflux condenser and drying tube in the presence of air. After 72 h, the purity had decreased (to 97.66% by GC), while the color number had risen to 176 APHA.

### Comparative Example 1

N-ethylpyrrolidone (250 ml, purity 99.69% by GC, Hazen color number: 7 APHA) was heated without additives to 100° C. in a 500 ml glass flask with attached reflux condenser and drying tube in the presence of air. After 72 h, the purity had decreased (to 97.77% by GC), while the color number had risen to 284 APHA.

### Example 3

N-ethylpyrrolidone (10 ml, purity 99.59% by GC, Hazen color number: 7 APHA) was admixed with dimethoxymethane (500 ppm). The mixture was heated to 100° C. in a 20 ml glass autoclave sealed gas-tight. After 72 hours (h), the purity had decreased (to 99.37% by GC), while the color number had risen to 22 APHA.

### Comparative Example 2

N-ethylpyrrolidone (10 ml, purity 99.59% by GC, Hazen color number: 7 APHA) was heated without additives to 100° C. in a 20 ml glass autoclave sealed gas-tight. After 72 hours (h), the purity had decreased (to 99.27% by GC), while the color number had risen to 32 APHA.

### Example 4

N-methylpiperidone (10 ml, purity 99.20% by GC, Hazen color number: 18 APHA) was admixed with methanol (500 ppm). The mixture was heated to 100° C. in a 20 ml glass autoclave sealed gas-tight. After 72 hours (h), the purity had decreased (to 98.82% by GC), while the color number had risen to 172 APHA.

### Comparative Example 3

N-methylpiperidone (10 ml, purity 99.20% by GC, Hazen color number: 18 APHA) was heated without additives to 100° C. in a 20 ml glass autoclave sealed gas-tight. After 72 hours (h), the purity had decreased (to 98.73% by GC), while the color number had risen to 197 APHA.

### Example 5

1,5-dimethyl-2-pyrrolidone (10 ml, purity 99.72% by GC, Hazen color number: 5 APHA) was admixed with methanol (500 ppm). The mixture was heated to 10° C. in a 20 ml glass autoclave sealed gas-tight. After 72 hours (h), the purity had decreased (to 99.53% by GC), while the color number had risen to 404 APHA.

### Comparative Example 4

1,5-dimethyl-2-pyrrolidone (10 ml, purity 99.72% by GC, Hazen color number: 5 APHA) was heated without additives to 100° C. in a 20 ml glass autoclave sealed gas-tight. After 72 hours (h), the purity had decreased (to 99.46% by GC), while the color number had risen to 669 APHA.

1: A process for preparing an N-alkyllactam with improved color quality, which comprises adding to the N-alkyllactam from 0.01 to 10% by weight of a C₁₋₁₀-alcohol or a compound which releases from 0.01 to 10% by weight of a C₁₋₁₀-alcohol.

2: The process according to claim 1 for preparing an N-alkyl-2-pyrrolidone or N-alkyl-2-piperidone with improved color quality.

3: The process according to claim 1 for preparing an N—(C₁₋₈-alkyl)-2-pyrrolidone or N—(C₁₋₈-alkyl)-2-piperidone with improved color quality.

4: The process according to claim 1 for preparing N-ethyl-2-pyrrolidone (NEP), 1,5-dimethyl-2-pyrrolidone, 1-ethyl-5-methyl-2-pyrrolidone, 1-methyl-2-piperidone or N-ethyl-ε-caprolactam (NEC) with improved color quality.

5: The process according to claim 1, wherein the C₁₋₁₀-alcohol is methanol, ethanol, n-propanol, isopropanol, n-butanol, n-pentanol, n-hexanol, n-heptanol, n-octanol, 1,2-ethylene glycol, 1,2-propanediol, 1,3-propanediol, glycerol, pentaerythritol and/or sorbitol.

6: The process according to claim 1, wherein the compound which releases the C₁₋₁₀-alcohol is an acetal, aminal or an orthoester.

7: The process according to claim 1 wherein the compound which releases the C₁₋₁₀-alcohol is dimethoxymethane, diethoxymethane, tetramethoxymethane, tetraethoxymethane, trimethyl orthoformate and/or triethyl orthoformate.

8: The process according to claim 1, wherein from 0.02 to 2% by weight of a C₁₋₁₀-alcohol or a compound which releases from 0.02 to 2% by weight of a C₁₋₁₀-alcohol is added to the N-alkyllactam.

9: The process according to claim 1, wherein from 0.03 to 1% by weight of a C₁₋₁₀-alcohol or a compound which releases from 0.03 to 1% by weight of a C₁₋₁₀-alcohol is added to the N-alkyllactam.

10: The process according to claim 1, which is performed at a temperature in the range from −20 to 400° C.

11: The process according to claim 1, which is performed at a temperature in the range from 10 to 250° C.

12: A mixture comprising at least 99.0% by weight of an N-alkyllactam and in the range from 100 to 5000 ppm by weight of a C₁₋₁₀-alcohol or of an acetal, aminal or of an orthoester which releases in the range from 100 to 5000 ppm by weight of a C₁₋₁₀-alcohol.

13: A mixture comprising at least 99.0% by weight of N-ethyl-2-pyrrolidone (NEP) and in the range from 100 to 5000 ppm by weight of methanol or 1,2-ethylene glycol.

14: A mixture comprising at least 99.0% by weight of N-ethyl-ε-caprolactam (NEC) and in the range from 100 to 5000 ppm by weight of methanol or 1,2-ethylene glycol.

15: A mixture comprising at least 99.0% by weight of 1,5-dimethyl-2-pyrrolidone and in the range from 100 to 5000 ppm by weight of methanol or 1,2-ethylene glycol.

16: A mixture comprising at least 99.0% by weight of 1-ethyl-5-methyl-2-pyrrolidone and in the range from 100 to 5000 ppm by weight of methanol or 1,2-ethylene glycol.

17: A mixture comprising at least 99.0% by weight of 1-methyl-2-piperidone and in the range from 100 to 5000 ppm by weight of methanol or 1,2-ethylene glycol.
